(19) United States
(12) Patent Application Publication
(10) Pub. No.: US 2024/0076320 A1
(43) Pub. Date: Mar. 7, 2024

(54) DOWNSTREAM PROCESS FOR PURIFICATION OF VIRAL PROTEINS WITH HYDROPHOBIC MEMBRANE DOMAIN FOR USE IN VACCINE COMPOSITIONS

(71) Applicant: Novavax, Inc., Gaithersburg, MD (US)

(72) Inventors: Timothy Hahn, Gaithersburg, MD (US); Sender Aspelund, Damascus, MD (US)

(21) Appl. No.: 18/458,789

(22) Filed: Aug. 30, 2023

Related U.S. Application Data
(60) Provisional application No. 63/374,278, filed on Sep. 1, 2022.

(51) Int. Cl. C07K 14/005; B01D 15/18; B01D 15/32; B01D 15/36; B01D 15/38; C07K 1/18; C07K 1/20; C07K 1/22; C07K 1/36

(52) U.S. Cl. CPC C07K 14/005; B01D 15/1871; B01D 15/327; B01D 15/363; B01D 15/3828; C07K 1/18; C07K 1/20; C07K 1/22; C07K 1/36; C12N 2760/16151; C12N 2760/16251; C12N 2760/18551

(57) ABSTRACT

The present invention is directed to methods of purifying viral proteins for use in vaccine compositions. The method includes a capture step and a polish step. The capture step includes passing a solution containing a protein over a hydrophobic interaction chromatography column and eluting a crude protein eluate from the column. The polish step includes passing the crude protein eluate over a ligand affinity chromatography column and recovering a first flow through intermediate, passing the first flow through intermediate over an anion exchange chromatography column and recovering a second flow through intermediate, and passing the second flow through intermediate over another ligand affinity chromatography column and recovering a purified protein eluate. The present invention also provides a purified protein having a hydrophobic membrane domain that is produced by a baculovirus expression system in cultured insect cells, wherein the purified protein has a purity of greater than 85%.

### CROSS-REFERENCE TO RELATED APPLICATIONS

This application claims priority to U.S. Provisional Patent Application No. 63/374,278, filed Sep. 1, 2022, the contents of which are hereby incorporated by reference herein in their entirety.

### FIELD OF THE INVENTION

The present disclosure relates generally to the field of purification of proteins. In particular, the present disclosure relates to methods of purifying viral proteins with a hydrophobic membrane domain for use in vaccine compositions.

### BACKGROUND

In recent years, the development of viral protein subunit vaccines has been prioritized due to the appealing safety and stability attributes of such vaccines as compared to live attenuated or inactivated vaccines. Rather than injecting a whole pathogen to trigger an immune response, subunit vaccines use specific isolated proteins from viral pathogens.

Efforts to develop protein subunit vaccines for common viral pathogens, including influenza, respiratory syncytial virus (RSV), and SARS-CoV-2 viruses, are continuing. For example, such vaccines may contain one or more proteins having a hydrophobic membrane domain. Examples of such proteins include recombinant influenza hemagglutinin (HA) proteins, recombinant SARS-CoV-2 spike (S) proteins, recombinant RSV fusion (F) proteins, or a combination thereof. These proteins can be generated through a baculovirus expression vector system (BEVS), in which the genetic instructions for the desired protein product are introduced into a baculovirus that is then used to infect insect cells. The insect cells produce the protein, which can then be harvested and purified for use in vaccine compositions.

To satisfy the requirements of use in pharmaceutical and therapeutic applications, isolated proteins that are used in protein subunit vaccines must undergo careful purification to remove any contaminants or impurities that may arise during protein production. Purification of the target proteins is often achieved by utilizing one or more chromatographic purification steps. However, because of similarities between the target proteins and baculovirus/host cell proteins, routine purification procedures based on particle size or charge are often inefficient or ineffective in removing host cell protein (HCP) impurities. In addition to impacting the purity of the target protein, HCP impurities can also negatively impact yield and process volume, making large scale manufacturing more complex, costly, and time consuming.

### SUMMARY

The present invention is directed to methods of purifying viral proteins for use in vaccine compositions, and specifically for purifying viral proteins having a hydrophobic membrane domain for use in protein subunit vaccines.

The present invention provides for a method of purifying a protein from a solution. The method includes a capture step (a) and a polish step (b). The capture step (a) includes: (i) passing the solution over a hydrophobic interaction chromatography column, and (ii) eluting a crude protein eluate from the hydrophobic interaction chromatography column. The polish step (b) includes: (i) passing the crude protein eluate obtained from the capture step over a ligand affinity chromatography column, (ii) recovering a first flow through intermediate from the ligand affinity chromatography column, (iii) passing the first flow through intermediate over an anion exchange chromatography column, (iv) recovering a second flow through intermediate from the anion exchange chromatography column, (v) passing the second flow through intermediate over a different ligand affinity chromatography column, and (vi) recovering a purified protein eluate from the ligand affinity chromatography column. The protein may be any protein having a hydrophobic membrane domain, including for example an HA protein derived from an influenza virus, an F protein derived from RSV, or an S protein derived from a SARS-CoV-2 virus.

The present invention also provides a purified protein having a hydrophobic membrane domain that is produced by a baculovirus expression system in cultured insect cells, wherein the purified protein has a purity of greater than 85%.

Protein purification methods in accordance with the present disclosure provide highly purified proteins for use in vaccine compositions while removing undesirable HCP impurities. The method provides highly purified protein substances while maintaining or increasing the yield of the purified target protein. The method reduces process volume, complexity, and cost, and can be scaled from laboratory to commercial settings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows unit operations for a method of purifying a viral protein having a hydrophobic membrane domain according to one embodiment of the disclosure.

FIG. 2 shows unit operations for a method of purifying a viral protein having a hydrophobic membrane domain according to one embodiment of the disclosure, including exemplary chromatography and filtration methods.

FIG. 3 shows unit operations for a method of purifying a viral protein having a hydrophobic membrane domain according to one embodiment of the disclosure, including exemplary resin selections for the exemplary chromatography methods.

FIG. 4 is a representative gel image showing the composition of the target protein-containing product following each chromatography step according to one embodiment of the disclosure.

FIG. 5 is a representative hydrophobic interaction chromatogram according to one embodiment of the present invention.

FIG. 6 is a representative GP64 affinity chromatogram according to one embodiment of the present invention.

FIG. 7 is a representative anion exchange chromatogram according to one embodiment of the present invention.

FIG. 8 is a representative lentil lectin chromatogram according to one embodiment of the present invention.

FIG. 9 depicts a process flow diagram for a method of purifying a viral protein having a hydrophobic membrane according to one embodiment of the disclosure.

### DETAILED DESCRIPTION

The present invention can be more readily understood by reading the following detailed description of the invention and study of the included examples.

As used herein, the following terms have the meanings ascribed to them unless specified otherwise.

The terms “comprise,” “comprising,” “include,” “including,” and “includes” when used in this specification and claims are intended to specify the presence of the stated materials, substances, features, integers, components, or steps, but they do not preclude the presence or addition of one or more other materials, substances, features, integers, components, steps, or combinations thereof.

The term “about” modifies the subject values, such that they are within an acceptable error range, as determined by one of ordinary skill in the art, which will depend in part on the limitations of the measurement system.

The articles “a” and “an” as used herein refer to “one or more” or “at least one,” unless otherwise indicated. That is, reference to any element or component of an embodiment by the indefinite article “a” or “an” does not exclude the possibility that more than one element or component is present.

The term “purified” as used herein refers to a target substance, such as a target protein, that has been isolated from a complex mixture that includes unwanted components. Where reference is made to a purified substance, such as a purified protein, the reference is intended to include both target substances that have been entirely isolated from a complex mixture and target substances that are substantially isolated, even where a pharmaceutically acceptable residual amount of impurities remain.

The term “purity” is used herein to refer to the percentage of a particular sample that is comprised of the target substance, such as a target protein. Unless otherwise specified the percentage purity of a substance is calculated using known densitometry image analysis techniques.

A “hydrophobic membrane domain” refers to a hydrophobic region of a protein's polypeptide chain that spans the viral membrane.

An “influenza hemagglutinin protein” or “HA protein” is an envelope glycoprotein present in the influenza virion that is responsible for viral attachment and penetration into the host cell. The influenza virus initiates infection by attachment of the virion surface HA protein to a sialic acid-containing cellular receptor. Following attachment, the HA protein undergoes conformational changes that lead to fusion of viral and host cell membranes followed by virus uncoating and M2-mediated release of M1 proteins from nucleocapsid-associated ribonucleoproteins (RNPs), which migrate into the cell nucleus for viral RNA synthesis. The membrane-distal “head” portion of the HA protein is a primary target of host cell antibodies that confer protective immunity to influenza viruses, and as a result the HA protein is a desirable antigen for use in commercial influenza vaccines. When reference is made to an “influenza hemagglutinin protein” or “HA protein” in the present disclosure, it should be understood that such reference may include wild type and/or modified HA proteins.

An “RSV fusion protein” or “F protein” is glycoprotein present on the surface of the RSV virion that controls the initial phase of RSV infection, and in particular causes the virion membrane to fuse with the target cell membrane. RSV initiates infection by attachment via interaction between the attachment glycoprotein (“G protein”) and a receptor surface of the host cell. Following attachment, the F protein undergoes conformational changes that lead to fusion of the viral and host cell membranes and expression of viral proteins. The RSV F protein is a primary target of host cell antibodies that confer protective immunity to RSV, and as a result the F protein is a desirable antigen for use in commercial RSV vaccines. When reference is made to an “RSV fusion protein” or “F protein” in the present disclosure, it should be understood that such reference may include wild type and/or modified F proteins.

A “SARS-CoV-2 spike protein” or “S protein” is a large structural protein present in the SARS-CoV-2 (COVID-19) virus that is responsible for viral attachment and penetration into the host cell. The SARS-CoV-2 virus initiates infection by attachment via interaction between the receptor-binding domain (S1 region) of the S protein and ACE2 receptor surface of the host cell. Following attachment, the S protein undergoes conformational changes that lead to fusion of viral and host cell membranes and expression of viral proteins. The SARS-CoV-2 S protein is a primary target of host cell antibodies that confer protective immunity to coronaviruses, and as a result the S protein is a desirable antigen for use in commercial COVID-19 vaccines. When reference is made to a “SARS-CoV-2 spike protein” or “S protein” in the present disclosure, it should be understood that such reference may include wild type and/or modified S proteins.

The term “baculovirus expression system” as used herein refers to a known platform for expressing recombinant proteins. To initiate baculovirus expression, a recombinant baculovirus is constructed by cloning the genes of interest into a transfer plasmid behind a strong promotor and surrounded by DNA homologous to the parent baculovirus. To generate the target protein, the recombinant baculovirus is scaled up and used to infect insect cells, which generate large quantities of the target protein for subsequent harvesting and purification. Insect cell lines for use in baculovirus expression systems are well known in the art.

The terms “host cell protein” or “HCP” as used herein refer to any non-target protein present in the baculovirus expression system. For example, HCPs may include recombinant baculovirus proteins, insect cells proteins, or combinations thereof.

The term “impurity” as used herein refers to any component that is not desired in the final product, including host cell DNA and HCPs.

The term “solution” as used herein refers to any homogenous mixture of two or more substances.

The term “capture step” as used herein refers to quickly isolating, concentrating, and stabilizing the target protein after harvest. During the capture step, clarified lysate containing the target protein is passed through a chromatography column, and the target protein is eluted into a significantly smaller volume for further downstream processing. In general, capture steps are designed to accommodate larger and more viscous process volumes, and therefore employ larger columns and resin particle sizes compared to other purification steps. The resin selected for the capture step should maximize retention and recovery of the target protein.

The term “polish step” as used herein refers to removing trace impurities from the target protein to provide a purified protein. During the polish step, the target protein-containing solution obtained from the capture step (referred to herein as a crude protein eluate) is passed through a series of chromatography columns designed to remove specific impurities. Polishing is achieved via the use of different separation principles over a series of chromatography columns to maximize removal of impurities with different characteristics.

The terms “hydrophobic interaction column” or “hydrophobic interaction chromatography column” as used herein are intended to refer to a chromatography column designed to separate molecules based on their hydrophobicity. The ligand and resin used in the HIC column should be selected to maximize the specificity of binding to the target protein and step yield.

The terms “ligand affinity column” or “ligand affinity chromatography column” as used herein are intended to refer to a chromatography column designed to separate molecules based on highly specific biological interactions between the ligand and the molecule of interest. The ligand and resin used in the ligand affinity column should be selected based on its impurity clearance and step yield.

The terms “anion exchange column” or “anion exchange chromatography column” as used herein are intended to refer to a type of chromatography column designed to separate molecules based on their net surface charge. An anion exchange column uses a positively charged ion exchange resin with an affinity for molecules having a net negative surface charge. The ligand and resin used in the anion exchange column should be selected based on its impurity clearance and step yield.

The term “protein eluate” as used herein is intended to refer to the target protein-containing product obtained from a chromatography step via elution. The term “crude protein eluate” is intended to refer to a protein eluate obtained through the capture step, whereas the term “purified protein eluate” is intended to refer to the purified protein product obtained after the final polish step.

The term “flow through intermediate” as used herein is intended to refer to the target protein-containing product that flows through a chromatography step while impurities remain bound to the column resin. The term “first flow through intermediate” is intended to refer to the flow through protein product obtained from the second chromatography step and the term “second flow through intermediate” is intended to refer to the flow through protein product obtained from the third chromatography step.

The term “filtration” as used herein is intended to refer to the separation process by which certain particles are removed from a solution based on particle size.

The terms “tangential flow filtration” or “TFF” as used herein are intended to refer to a unit operation used for clarifying, concentrating, and/or purifying proteins via a pressure driven filtration process. During TFF, the feed solution is passed tangentially along the surface of a membrane while an applied pressure forces a portion of the fluid through the membrane. Particles that are too large to pass through the membrane pores are retained on the upstream side of the membrane (referred to as the retentate), and can be recycled through the TFF process as desired. Particles that are small enough to pass through the membrane pores (the filtrate) are collected and either disposed of or passed on to the next unit operation.

The same TFF unit operation can be used for micro- or ultrafiltration and diafiltration, which decreases process time and avoids product loss. When both micro- or ultrafiltration and diafiltration are preformed in the same unit operation, the diafiltration step is initiated by feeding the diafiltration solution (i.e. water or a buffer) into the recycled retentate stream and recirculating the solution across the membrane filter. In this way, the TFF process can be used to first concentrate the target protein (via microfiltration or ultrafiltration) and then diafilter the protein by exchanging the buffer solution, decreasing salt concentration, and/or removing solvents or other additives.

The term “viral filtration” as used herein refers to a filtration process designed to remove endogenous or adventitious viruses from the target protein solution. Viral filtration is a direct flow filtration process that employs a membrane filter with a size exclusion mechanism that allows target proteins to pass through the membrane without adsorption or denaturation while efficiently capturing viruses.

The target proteins that are subjected to the purification method of the present invention are typically produced by recombinant expression in host cells, and in particular via a baculovirus expression system. Target proteins may be expressed in any suitable host cell, for example in insect cells. Non-limiting examples of insect cells are Spodoptera frugiperda (Sf) cells (e.g Sf9, Sf21, Sf22a), Trichoplusiani cells, and Drosophila S2 cells.

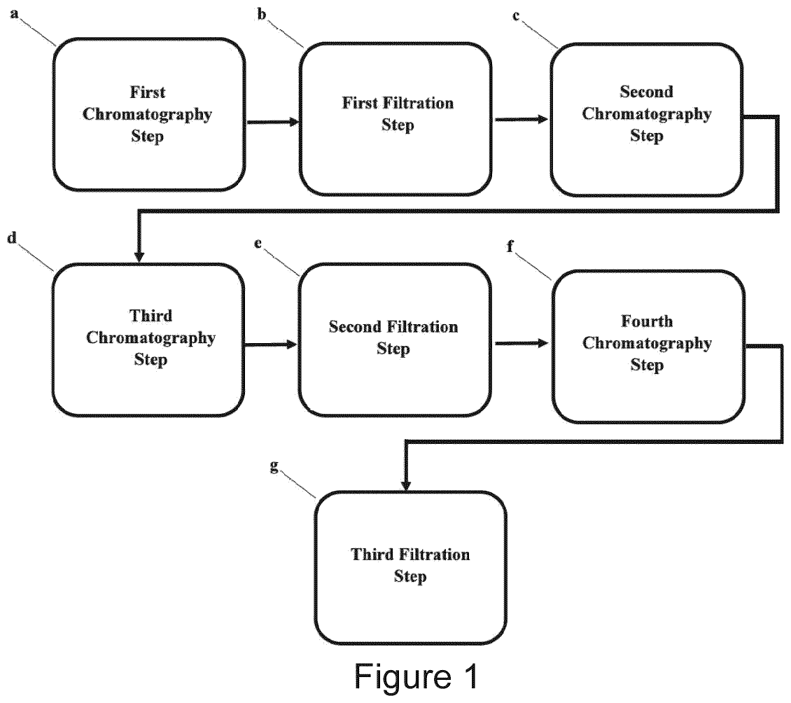

Following host cell expression and expansion, the host cells are harvested via lysis in the bioreactor and clarified via centrifugation. The resulting clarified lysate solution is then subjected to the method for protein purification provided in the present invention. Referring to FIG. 1, the method for purifying target proteins may include a first chromatography step a, a first filtration step b, a second chromatography step c, a third chromatography step d, a second filtration step e, a fourth chromatography step f, and a third filtration step g.

It should be understood that certain steps may be added or modified to the method of the present invention so long as the primary steps described in FIG. 1 are employed. For example, the method may further include a virus inactivation step that serves to inactivate endogenous or adventitious viruses that may be present in the clarified lysate solution. As another example, additional filtration steps may be included immediately prior to each chromatography step and/or filtration step to remove undesirable particulates that may adsorb particles of interest, result in instrument downtime, or decrease column or filter life.

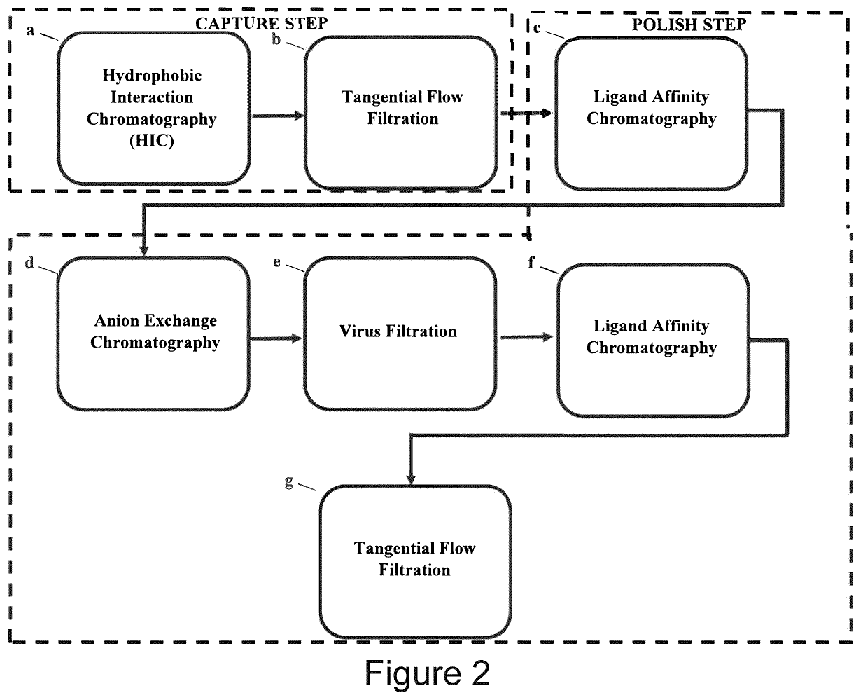

As shown in FIG. 2, in certain embodiments the method for purifying a target protein employs chromatography steps that operate using different separation principles to maximize removal of impurities with different characteristics. As described in more detail below, the particular order of the chromatography steps of the present invention serve to maximize process volume and yield while minimizing process cost and complexity.

As further shown in FIG. 2, in certain embodiments the first chromatography step a is a capture step that employs hydrophobic interaction chromatography. During this step, the column is equilibrated with high-salt buffer and then the clarified lysate solution is loaded onto the column. The high salt buffer promotes interaction between the target proteins in the clarified lysate solution and the hydrophobic regions of the resin. Exemplary equilibration buffers include, but are not limited to, sulfate buffers, phosphate buffers, citrate buffers, TRIS buffers, and histidine buffers. It should be understood that the particular salt and salt concentration of the high salt buffer should be optimized so as to minimize protein precipitation while maximizing ligand-protein binding. Exemplary salts for use in the buffer solution include, but are not limited to, sodium sulphate, sodium phosphate, potassium sulphate, potassium phosphate, ammonium sulphate, and ammonium phosphate. The salt concentration required to promote binding is inversely proportional to the hydrophobicity of the molecule, and therefore the salt concentration of the equilibration buffer may be adjusted according to the degree of hydrophobicity exhibited by the target protein. After loading the clarified lysate solution, the column is re-equilibrated with the high salt buffer and then washed to remove weakly bound impurities. The target proteins are then eluted with a salt-free buffer, and the resulting crude protein eluate is collected from the column.

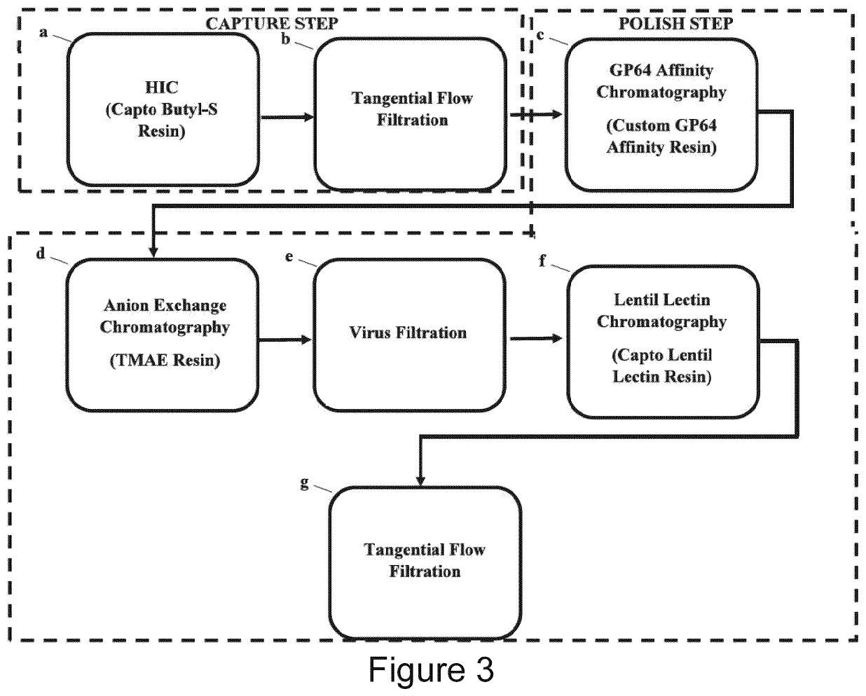

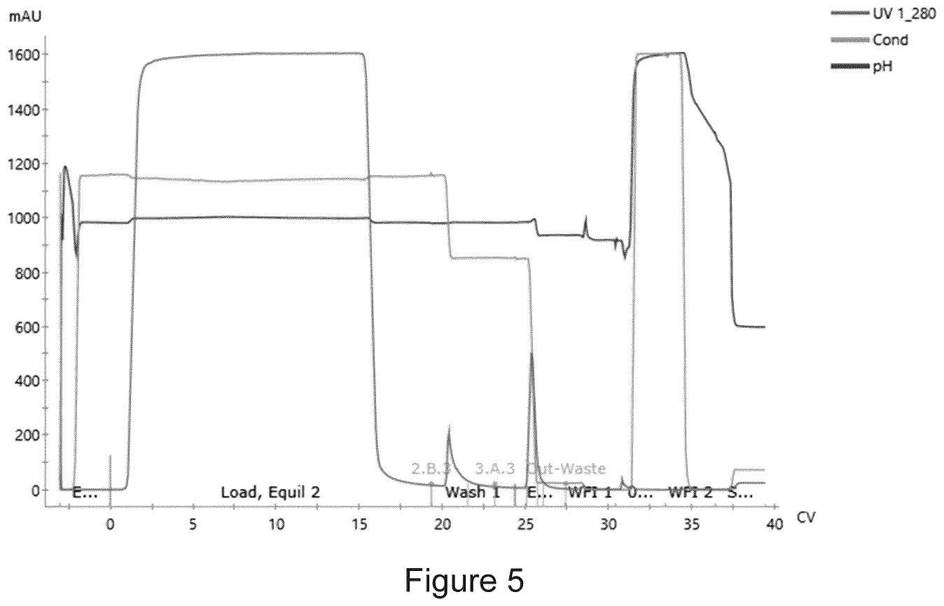

As shown in FIG. 3, in certain embodiments the first chromatography step a is a hydrophobic interaction chromatography step that employs a capture resin with high specificity on binding to the target protein, thereby promoting increased step yield across the column. For example, where the target protein is an HA protein, a Butyl-S Sepharose 6 Fast Flow resin may be used to promote binding specificity. FIG. 5 provides a representative chromatogram showing recovery of an HA protein (B/Phuket LN:1203-105 (label V)) during first chromatography step a where the first chromatography step a is a hydrophobic interaction chromatography step performed using a Butyl-S Sepharose 6 Fast Flow resin. While the hydrophobic interaction chromatography step operates to effectively separate any protein having a hydrophobic membrane domain, the particular resin for use in purifying the target protein may be optimized to promote binding specificity.

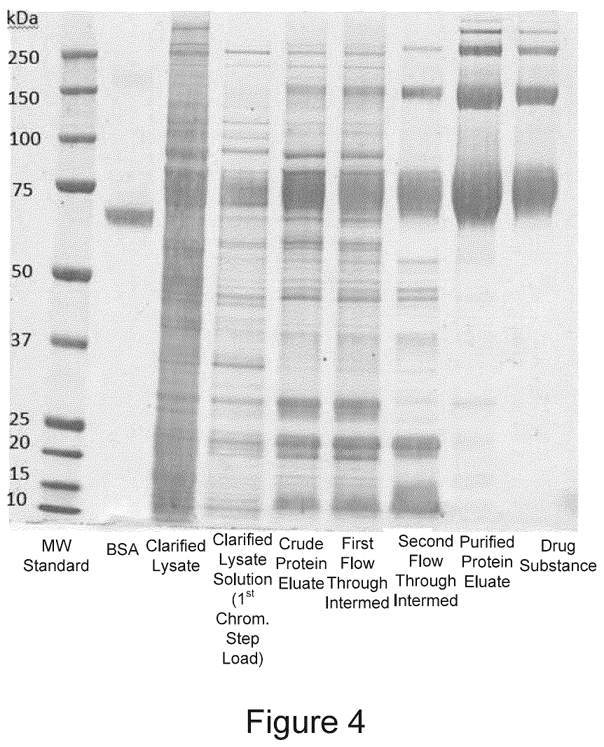

Given the high specificity on binding to the target protein, it is possible to pass large volumes of clarified lysate solution through the hydrophobic interaction column used in first chromatography step a while achieving very high target protein recovery. The high purity and recovery of the crude protein eluate versus the load solution is shown in FIG. 4. Because the target protein is eluted from the column as a crude protein eluate, it is also possible to significantly reduce the process volume over the first chromatography step a. The ability to process a high-volume clarified lysate solution advantageously allows cell harvest to be achieved via lysis in the bioreactor rather than via multiple centrifugation passes, which do not easily scale up to industrial production capacity and can result in loss of more than half of the target protein before it even enters the purification process. As a result, the first chromatography step a of the present invention may advantageously increase process yield and/or decrease downstream process volume.

Returning to FIG. 2, in certain embodiments the second, third, and fourth chromatography steps are polish steps that are designed to remove trace impurities from the target protein. For example, the second chromatography step c and fourth chromatography step f are polish steps that employ ligand affinity chromatography to separate impurities from the target protein, while the third chromatography step d employs an anion exchange column. As shown in FIG. 3, the affinity resins employed in the second chromatography step c and fourth chromatography step f may be varied to achieve separation from different types of impurities.

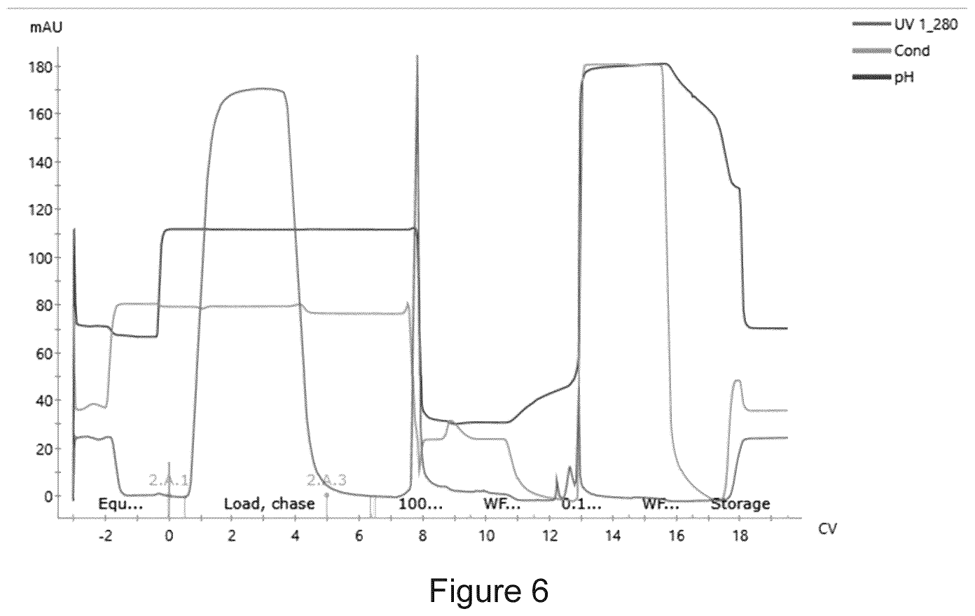

As is also shown in FIG. 3, in certain embodiments the second chromatography step c is a ligand affinity column that employs a custom GP64 affinity resin. During this step, the crude protein eluate obtained from first chromatography step a is loaded onto the equilibrated column and chased with equilibration buffer. Because the selected resin has a high affinity for GP64 proteins, the GP64 proteins bind to the resin and the target proteins are collected as a first flow through intermediate. By employing an affinity resin with exceptionally high specificity for GP64 proteins early in the polishing step, second chromatography step c yields a high purity first flow through intermediate without GP64 proteins that problematically co-purify with the target protein in prior art processes. As a result, as shown in FIG. 4, the third chromatography step d and fourth chromatography step f are supplied with cleaner load material, which improves step yield and unit capacity. FIG. 6 provides a representative chromatogram showing recovery of an HA protein (B/Phuket LN:1203-105 (label V)) during second chromatography step c where the second chromatography step c is a GP64 affinity column.

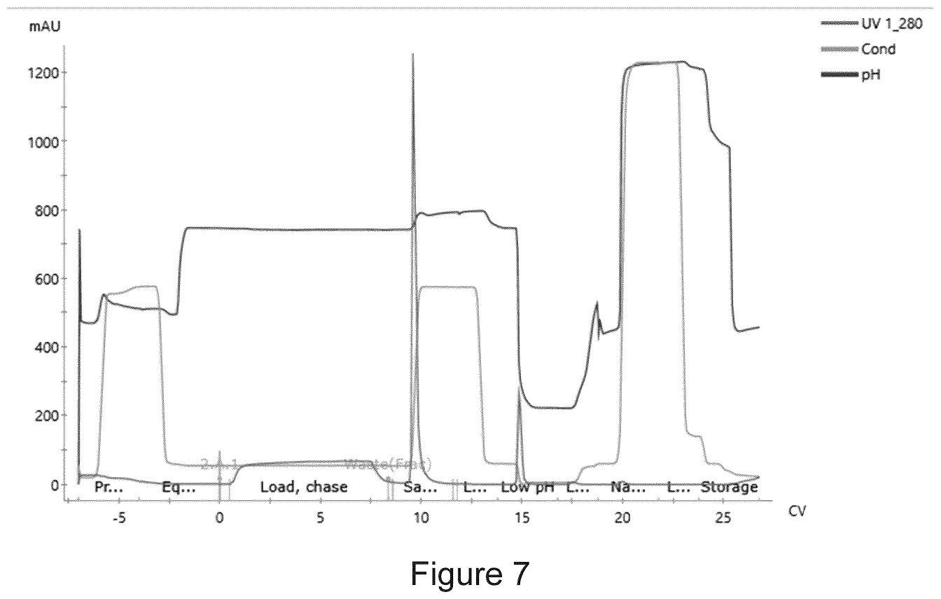

As is further shown in FIGS. 2-3, in certain embodiments the third chromatography step d is an anion exchange column that employs a trimethylaminoethyl (TMAE) resin. During this step, the first flow through intermediate obtained from the second chromatography step c is loaded onto the equilibrated column and chased with an equilibration buffer. The negatively charged impurities bind to the resin, while the target proteins are collected as a second flow through intermediate. FIG. 7 provides a representative chromatogram showing recovery of an HA protein (B/Phuket LN:1203-105 (label V)) during third chromatography step d where the third chromatography step d is an anion exchange column that employs a TMAE resin.

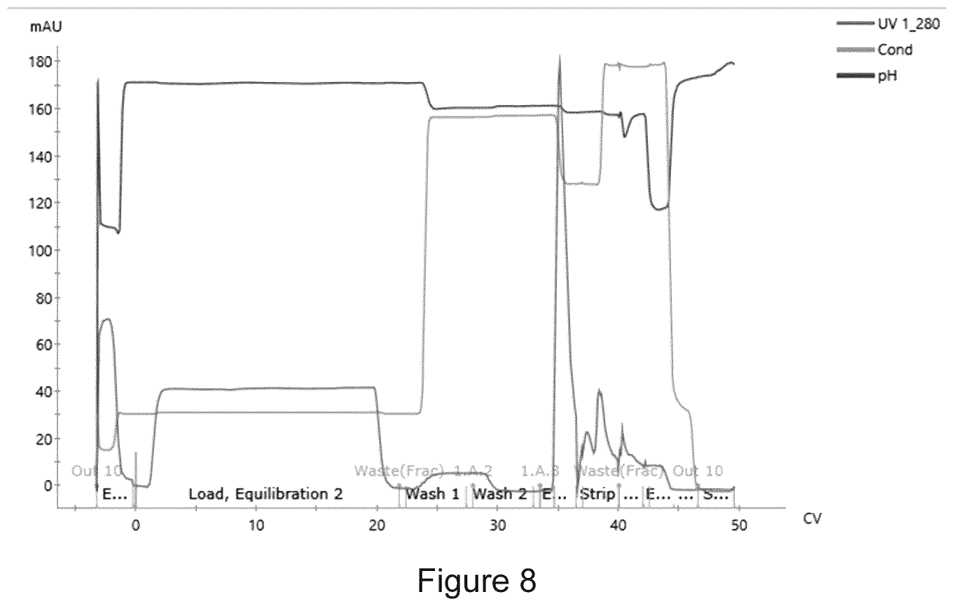

As is further shown in FIG. 3, in certain embodiments the fourth chromatography step f is a ligand affinity column that employs a lentil lectin affinity resin. During this step, the second flow through intermediate obtained from the third chromatography step d is loaded onto the equilibrated column. The lentil lectin affinity resin tightly binds the target protein through glycosylation while the column undergoes dual washes to remove the remaining impurities. Following the washes, the target proteins are eluted from the column and collected as a purified protein eluate. As shown in FIG. 4, the purified protein eluate is nearly free of impurities and closely resembles the desired composition of the final drug substance. FIG. 8 provides a representative chromatogram showing recovery of an HA protein (B/Phuket LN:1203-105 (label V)) during fourth chromatography step f where the fourth chromatography step f is a lentil lectin affinity column.

In certain embodiments, the fourth chromatography step f may include a detergent exchange during elution. By exchanging detergents, it is possible to encourage protein attributes that are desirable for use in the vaccine composition. For example, an elution buffer containing a non-ionic detergent may be applied to the column during fourth chromatography step f to facilitate the formation of the target protein into nanoparticles. In certain embodiments, the non-ionic detergent may be selected from the group consisting of polysorbate-20 (PS-20), polysorbate-40 (PS-40), polysorbate-60 (PS-60), polysorbate-65 (PS-65), and polysorbate-80 (PS-80).

As would be understood by a person of ordinary skill in the art, following execution of each chromatography step the column can be flushed, stripped and/or cleaned, and stored for repeat use.

Returning to FIG. 1, the method for purifying proteins may further include a first filtration step b after first chromatography step a, a second filtration step e after third chromatography step d, and a third filtration step g after fourth chromatography step f. As shown in FIGS. 2-3, in certain embodiments the first filtration step b and third filtration step g employ tangential flow filtration and the second filtration step e is a virus filtration step.

During the first filtration step b, the crude protein eluate is concentrated in the filtrate through use of a filtration membrane sized to allow removal of the salts from the first chromatography step a while retaining the target protein in the retentate. The crude protein eluate is concentrated, diafiltered to remove salts, and chased with a buffer to recover the crude protein eluate for loading into the second chromatography step c. Because the isoelectric points for viral proteins having hydrophobic membrane domains may vary depending on the strain or variant of the particular virus, it may be necessary to vary the pH and/or salt concentration of the solution during first filtration step b so as to prevent the target protein from binding to the anion exchange resin.

During the third filtration step g, the purified protein eluate is concentrated in the retentate while smaller impurities are passed through the membrane filter into the filtrate and removed from the process. Following concentration, the purified protein eluate is diafiltered, diluted to a target concentration, and spiked with a non-ionic detergent to facilitate the nanoparticle formation desired in the vaccine composition. It should be noted that where a detergent exchange is employed during the fourth chromatography step to introduce the non-ionic detergent, the non-ionic detergent will also be concentrated in the retentate during third filtration step g.

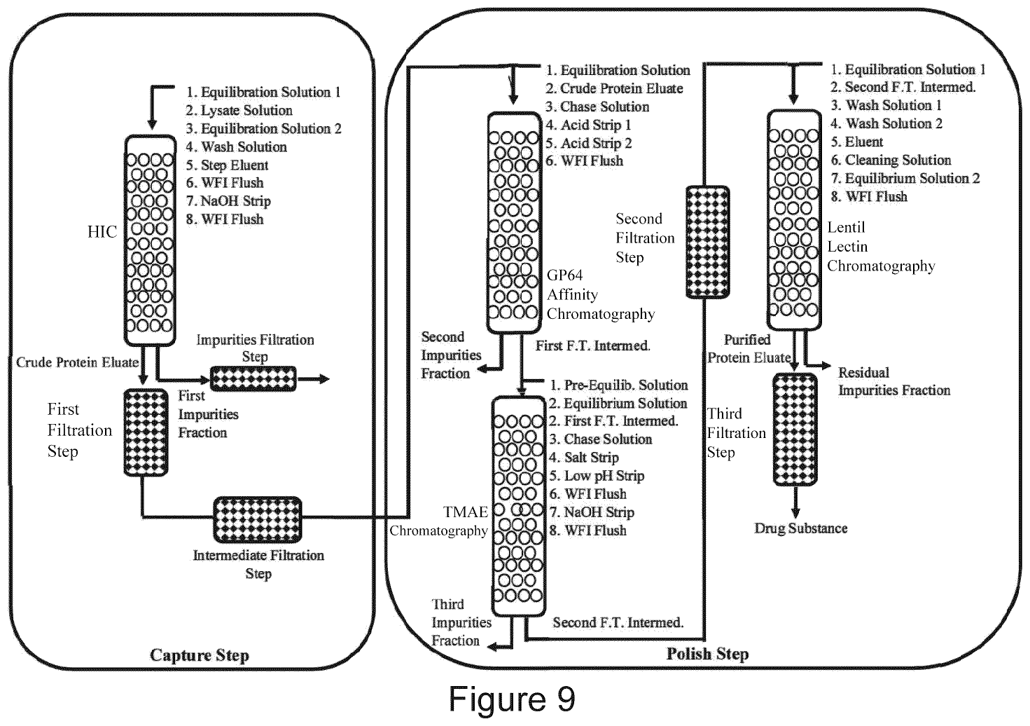

A process flow diagram depicting one embodiment of the method of purifying proteins described above is provided herein as FIG. 9. It should be understood that the particular equilibration solutions, wash solutions, eluents, and chase solutions described herein and in FIG. 9 may be varied according to the characteristics of the target protein and/or the particular impurities created during the host cell expression process.

It should be understood that certain steps may be added or modified to the method previously described so long as the primary steps described in FIG. 1 are employed. For example, as shown in FIG. 9, the method may further include an intermediate filtration step and/or an impurities filtration step. As another example, the method may include a virus inactivation step that serves to inactivate endogenous or adventitious viruses that may be present in the clarified lysate solution and/or additional filtration steps prior to each chromatography step to remove undesirable particulates that may adsorb particles of interest, result in instrument downtime, or decrease column or filter life.

### Embodiments

An aspect of the present invention relates to a method of purifying a protein from a solution. The method includes (a) a capture step and (b) a polish step. The capture step (a) includes: (i) passing the solution over a hydrophobic interaction chromatography column, and (ii) eluting a crude protein eluate from the hydrophobic interaction chromatography column. The polish step (b) includes: (i) a second chromatography step wherein the crude protein eluate obtained from the capture step is passed over a ligand affinity chromatography column, (ii) recovering a first flow through intermediate from the ligand affinity chromatography column, (iii) a third chromatography step wherein the first flow through intermediate is passed over an anion exchange chromatography column, (iv) recovering a second flow through intermediate from the anion exchange chromatography column, (v) a fourth chromatography step wherein the second flow through intermediate is passed over a second ligand affinity chromatography column, and (vi) recovering a purified protein eluate from the ligand affinity chromatography column. The protein may be an HA protein derived from an influenza virus, an S protein derived from a SARS-CoV-2 virus, or an F protein derived from RSV.

The present invention also provides a purified protein having a hydrophobic membrane domain that is produced by a baculovirus expression system in cultured insect cells, wherein the purified protein has a purity of greater than 85%.

The present invention also provides a purified HA protein produced by a baculovirus expression system in cultured insect cells, wherein the purified influenza HA protein has a purity of greater than 85%.

The present invention also provides a purified F protein produced by a baculovirus expression system in cultured insect cells, wherein the purified RSV F protein has a purity of greater than 85%.

The specific embodiments describing the components, materials, ranges, values, and steps provided below are for illustration purposes only, and do not otherwise limit the scope of the disclosed subject matter, as defined by the claims.

In various embodiments, the hydrophobic interaction chromatography column employs a resin with high specificity for the target protein. For example, the hydrophobic interaction chromatography column may comprise a Butyl-S resin.

In various embodiments, the ligand affinity column of the second chromatography step employs a resin with high specificity for GP64 proteins. For example, the ligand affinity column of the second chromatography step may comprise a GP64 affinity resin.

In various embodiments, the anion exchange column employs a resin for effectively attracting negatively-charged impurities. For example, the anion exchange column may comprise a TMAE resin.

In various embodiments, the ligand affinity column of the fourth chromatography step employs a resin with high specificity for the target protein. For example, the ligand affinity column of the fourth chromatography step may comprise a lentil lectin resin.

In various embodiments, the target protein in the purified protein product is an HA protein derived from an influenza virus. The influenza virus may be one or more influenza virus subtypes selected from the group consisting of: Influenza A Group 1, Influenza A Group 2, and Influenza B.

In various embodiments, the target protein in the purified protein product is an F protein derived from a RSV. The RSV may be one or more RSV subtypes selected from the group consisting of: respiratory syncytial virus subtype A (RSV-A) and respiratory syncytial virus subtype B (RSV-B).

In various embodiments, the method for purifying the protein from the solution further comprises one or more filtration steps. For example, the capture step (a) may further include a first filtration step after the hydrophobic interaction chromatography column. In certain embodiments the first filtration step may include tangential flow filtration. As another example, polish step (b) may further include a second filtration step after the anion exchange chromatography step and a third filtration step after the final ligand affinity chromatography step. In certain embodiments the second filtration step may include viral filtration and the third filtration step may include tangential flow filtration.

### Enumerated Embodiments

Specific enumerated Embodiments 1 to 18 provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.

Embodiment 1: A method including a capture step (a) that includes (i) passing the solution over a hydrophobic interaction chromatography column, and (ii) eluting a crude protein eluate from the hydrophobic interaction chromatography column, and a polish step (b) that includes (i) passing the crude protein eluate obtained from the capture step over a second chromatography step including a ligand affinity chromatography column, (ii) recovering a first flow through intermediate from the ligand affinity chromatography column, (iii) passing the first flow through intermediate over an anion exchange chromatography column, (iv) recovering a second flow through intermediate from the anion exchange chromatography column, (v) passing the second flow through intermediate over a fourth chromatography step including a ligand affinity chromatography column, and (vi) recovering a purified protein eluate from the ligand affinity chromatography column.

Embodiment 2: The method of embodiment 1, wherein the protein is a protein having a hydrophobic membrane domain.

Embodiment 3. The method of any one of embodiments 1-2, wherein the protein is derived from an influenza virus, RSV, or a SARS-CoV-2 virus.

Embodiment 4: The method of any one of embodiments 1-3, wherein the protein is an HA protein derived from an influenza virus.

Embodiment 5: The method of embodiment 4, wherein the influenza virus may be one or more influenza virus subtypes selected from the group consisting of: Influenza A Group 1, Influenza A Group 2, and Influenza B.

Embodiment 6: The method of any one of embodiments 1-3, wherein the protein is an F protein derived from an RSV.

Embodiment 7: The method of embodiment 6, wherein the RSV may be one or more RSV subtypes derived from the group consisting of: respiratory syncytial virus subtype A (RSV-A) and respiratory syncytial virus subtype B (RSV-B).

Embodiment 8: The method of any one of embodiments 1-7, wherein the hydrophobic interaction chromatography column includes a Butyl-S resin.

Embodiment 9: The method of any one of embodiments 1-8, wherein the ligand affinity column of the second chromatography step includes a GP64 affinity resin.

Embodiment 10: The method of any one of embodiments 1-9, wherein the anion exchange column includes a trimethylaminoethyl (TMAE) resin.

Embodiment 11: The method of any one of embodiments 1-10, wherein the ligand affinity column of the fourth chromatography step includes a lentil lectin resin.

Embodiment 12: The method of any one of embodiments 1-11, wherein the method for purifying the protein includes one or more filtration steps.

Embodiment 13: The method of embodiment 12, wherein the method includes a first filtration step after the hydrophobic interaction chromatography column.

Embodiment 14: The method of embodiment 13, wherein the first filtration step is a tangential flow filtration step.

Embodiment 15: The method of any one of embodiments 13-14, wherein the method includes a second filtration step after the anion exchange chromatography column.

Embodiment 16: The method of embodiment 15, wherein the second filtration step is a viral filtration step.

Embodiment 17: The method of any one of embodiments 15-16, wherein the method includes a third filtration step after the lentil lectin chromatography column of the fourth chromatography step.

Embodiment 18: The method of embodiment 17, wherein the third filtration step is a tangential flow filtration step.

Embodiment 19: A purified protein having a hydrophobic membrane domain that is produced by a baculovirus expression system in cultured insect cells, wherein the purified protein has a purity of greater than 85%.

Embodiment 20: A purified HA protein produced by a baculovirus expression system in cultured insect cells, wherein the purified influenza HA protein has a purity of greater than 85%.

Embodiment 21: A purified F protein produced by a baculovirus expression system in cultured insect cells, wherein the purified RSV F protein has a purity of greater than 85%.

### Examples

Example 1: The following example provides process parameters for a capture step employing hydrophobic interaction chromatography with Butyl-S Sepharose 6 Fast Flow resin according to one embodiment of the present invention.

| Step | Parameter | Units | Range |
|---|---|---|---|
| Equili- | Buffer | 25 mM Tris, 700 mM sodium sulfate, 0.02% | |
| bration | | (w/v) 15-S-9, pH 8.0 | |
| | Volume | CV | ≤5 |
| | Residence | Minutes | ≥4 |
| | Time | | |
| Load | Buffer | 25 mM Tris, 1.2M Sodium Sulfate, pH 8.0 | |
| | Load Sample | Clarified Lysate Solution | |
| | Column Load | mg/mL resin | ≤55 |
| | Residence | Minutes | ≥4 |
| | Time | | |
| Equili- | Buffer | 25 mM Tris, 700 mM sodium sulfate, 0.02% | |
| bration | | (w/v) 15-S-9, pH 8.0 | |
| | Volume | CV | ≤5 |
| | Residence | Minutes | ≥4 |
| | Time | | |
| Wash | Buffer | 25 mM Tris, 450 mM sodium sulfate, | |
| | | 0.023% (w/v) 15-S-9, pH 8.0 | |
| | Volume | CV | ≥5 |
| | Residence | Minutes | ≥4 |
| | Time | | |
| Elution | Buffer | 25 mM Tris, 0.02% (w/v) 15-S-9, pH 8.0 | |
| | Start | OD₂₈₀ | ≤0.25 |
| | Collection | | |
| | Criteria | | |
| | Residence | Minutes | ≥4 |
| | Time | | |

Example 2: The following example provides process parameters for a capture step employing tangential flow filtration with a Repligen TangenX® SIUS® 50 kDa cassette membrane according to one embodiment of the present invention.

| Step | Parameter | Units | Range |
|---|---|---|---|
| Equilibration | Buffer | 25 mM Tris, 50 mM sodium chloride, | |
| | | 0.02% (w/v) 15-S-9, pH 8.0 | |
| | Feed Flow Rate | L/m²/h | ≤360 |
| | Transmembrane | Psid | ≤35 |
| | Pressure | | |
| | Volume | L/m² | ≥10 |
| Concentration | Intermediate | Crude Protein Eluate | |
| | Feed Flow Rate | L/m²/h | ≤360 |
| | Transmembrane | psid | 10-25 |
| | Pressure | | |
| | Membrane | g/m² | ≤500 |
| | Protein Load | | |
| | Target | Fold | ≤4 |
| | Concentration | | |
| | Factor | | |
| Diafiltration | Buffer | 25 mM Tris, 50 mM sodium chloride, | |
| | | 0.02% (w/v) 15-S-9, pH 8.0 | |
| | Feed Flow Rate | L/m²/h | ≤360 |
| | Transmembrane | psid | 10-25 |
| | Pressure | | |
| | Diavolumes | DV | ≥4 |
| Recovery | Buffer | 25 mM Tris, 50 mM sodium chloride, | |
| | | 0.02% (w/v) 15-S-9, pH 8.0 | |
| | Feed Flow Rate | L/m²/h | ≤360 |

Example 3: The following example provides process parameters for an intermediate filtration step that employees viral filtration with a Asahi Kasei Planova 20N filter according to one embodiment of the present invention.

| Step | Parameter | Units | Range |
|---|---|---|---|
| Equilibration | Buffer | 25 mM Tris, 50 mM sodium chloride, | |
| | | 0.02% (w/v) 15-S-9, pH 8.0 | |
| | Volume, priming | L/m² | ≥1.0 |
| | Volume, permeate | L/m² | ≥3.0 |
| Load | Intermediate | Crude Protein Eluate | |
| | Load Filter | 0.45 μm/0.2 μm | |
| | Volumetric Load | L/m² | ≤140 |
| Chase | Buffer | 25 mM Tris, 50 mM sodium chloride, | |
| | | 0.02% (w/v) 15-S-9, pH 8.0 | |
| | Volume | L/m² | 15-25 |

Example 4: The following example provides process parameters for a polish step employing GP64 affinity chromatography with Repligen/Navigo GP64 affinity resin according to one embodiment of the present invention.

| Step | Parameter | Units | Range |
|---|---|---|---|
| Equilibration | Buffer | 25 mM Tris, 50 mM sodium chloride, | |
| | | 0.02% (w/v) 15-S-9, pH 8.0 | |
| | Volume | CV | ≥3 |
| | Residence Time | Minutes | ≥4 |
| Load | Load Sample | Crude Protein Eluate | |
| | Column Load | mg/mL resin | ≤50 |
| | Start Collection | OD₂₈₀ | ≤0.25 |
| | Criteria | | |
| | Residence Time | Minutes | ≥4 |
| Chase | Buffer | 25 mM Tris, 50 mM sodium chloride, | |
| | | 0.02% (w/v) 15-S-9, pH 8.0 | |
| | End Collection | OD₂₈₀ | ≤0.25 |
| | Criteria | | |
| | Residence Time | Minutes | ≥4 |

Example 5: The following example provides process parameters for a polish step employing anion exchange chromatography with Fractogel EMD TMAE (M) resin according to one embodiment of the present invention.

| Step | Parameter | Units | Range |
|---|---|---|---|
| Pre- | Buffer | 1M sodium chloride | |
| Equilibration | Volume | CV | ≥3 |
| | Residence Time | Minutes | ≥4 |
| Equilibration | Buffer | 25 mM Tris, 50 mM sodium chloride, | |
| | | 0.02% (w/v) 15-S-9, pH 8.0 | |
| | Volume | CV | ≥4 |
| | Residence Time | Minutes | ≥4 |
| Load | Load Sample | First Flow Through Intermediate | |
| | Column Load | mg/mL resin | ≤5 |
| | Start Collection | OD₂₈₀ | ≤0.25 |
| | Criteria | | |
| | Residence Time | Minutes | ≥4 |
| Chase | Buffer | 25 mM Tris, 50 mM sodium chloride, | |
| | | 0.02% (w/v) 15-S-9, pH 8.0 | |
| | End Collection | OD₂₈₀ | ≤0.25 |
| | Criteria | | |
| | Residence Time | Minutes | ≥4 |

Example 6: The following example provides process parameters for a polish step employing viral filtration with a Asahi Kasei Planova 20N filter according to one embodiment of the present invention.

| Step | Parameter | Units | Range |
|---|---|---|---|
| Equilibration | Buffer | 25 mM Tris, 50 mM sodium chloride, | |
| | | 0.02% (w/v) 15-S-9, pH 8.0 | |
| | Volume, priming | L/m² | ≥1.0 |
| | Volume, permeate | L/m² | ≥3.0 |
| Load | Intermediate | Second Flow Through Intermediate | |
| | Load Filter | 0.45 μm/0.2 μm | |
| | Volumetric Load | L/m² | ≤140 |
| Chase | Buffer | 25 mM Tris, 50 mM sodium chloride, | |
| | | 0.02% (w/v) 15-S-9, pH 8.0 | |
| | Volume | L/m² | 15-25 |

Example 7: The following example provides process parameters for a polish step employing lentil lectin chromatography with Capto Lentil Lectin resin according to one embodiment of the present invention.

| Step | Parameter | Units | Range |
|---|---|---|---|
| Equilibration | Buffer | 25 mM Tris, 50 mM sodium chloride, | |
| | | 0.02% (w/v) 15-S-9, pH 8.0 | |
| | Volume | CV | ≥3 |
| | Residence Time | Minutes | ≥5 |
| Load | Load Sample | Second Flow Through Intermediate | |
| | Column Load | mg/mL resin | ≤2.5 |
| | Residence Time | Minutes | ≥5 |
| Wash 1 | Buffer | 25 mM Tris, 500 mM sodium chloride, | |
| | | 0.1% (w/v) PS-80, pH 7.5 | |
| | Volume | CV | ≥5 |
| | Residence Time | Minutes | ≥5 |
| Wash 2 | Buffer | 25 mM Tris, 500 mM sodium chloride, | |
| | | 0.01% (w/v) PS-80, pH 7.5 | |
| | Volume | CV | ≥5 |
| | Residence Time | Minutes | ≥5 |
| Elution | Buffer | 25 mM sodium chloride, 0.5M MMP, | |
| | | 0.01% (w/v) PS-80, pH 7.5 | |
| | Start Collection | OD₂₈₀ | ≤0.25 |
| | Criteria | | |
| | Residence Time | Minutes | ≥8 |

Example 8: The following example provides process parameters for a polish step employing tangential flow filtration with a Repligen TangenX® SIUS® 50 kDa cassette membrane according to one embodiment of the present invention.

| Step | Parameter | Units | Range |
|---|---|---|---|
| Equilibration | Buffer | 25 mM Tris, 500 mM sodium chloride, | |
| | | 0.01% (w/v) PS-80, pH 7.5 | |
| | Feed Flow | L/m²/h | ≤360 |
| | Rate | | |
| | Trans- | Psid | ≤35 |
| | membrane | | |
| | Pressure | | |
| | Volume | L/m² | ≥10 |
| Concentration | Intermediate | Purified Protein Eluate | |
| | Feed Flow | L/m²/h | ≤360 |
| | Rate | | |
| | Trans- | psid | ≤35 |
| | membrane | | |
| | Pressure | | |
| | Membrane | g/m² | ≤500 |
| | Protein Load | | |
| | Target Protein | mg/mL | 2.5 |
| | Concentration | | |
| Diafiltration | Buffer | 25 mM Tris, 150 mM sodium chloride, | |
| | | 100 mM arginine, 5% (w/v) trehalose, | |
| | | pH 7.5 | |
| | Feed Flow | L/m²/h | ≤360 |
| | Rate | | |
| | Trans- | psid | ≤35 |
| | membrane | | |
| | Pressure | | |
| | Diavolumes | DV | ≥6 |
| Recovery | Buffer | 25 mM sodium phosphate, 150 mM | |
| | | sodium chloride, 100 mM arginine, | |
| | | 5% (w/v) trebalose, pH 7.5 | |
| | Feed Flow | L/m²/h | ≤360 |
| | Rate | | |
| | Target Protein | mg/mL | 1.5 |
| | Concentration | | |
| PS-80 Spike | Buffer | 10% (w/v) PS-80 | |
| | Target PS-80 | % (w/v) | 0.1 |
| | Concentration | | |

What is claimed is:

1. A purified influenza hemagglutinin (HA) protein produced by a baculovirus expression system in cultured insect cells, wherein the purified influenza HA protein has a purity of greater than 85 wt %.

2. A method for purifying a protein from a solution comprising:
(a) a capture step comprising:
(i) passing the solution over a first chromatography column, wherein the first chromatography column is a hydrophobic interaction column; and
(ii) eluting a crude protein eluate from the first chromatography column; and
(b) a polish step comprising:
(i) passing the crude protein eluate obtained from step (a) over a second chromatography column, wherein the second chromatography column is a ligand affinity column;
(ii) recovering a first flow through intermediate from the second chromatography column;
(iii) passing the first flow through intermediate obtained from step (b)(ii) over a third chromatography column, wherein the third chromatography column is an anion exchange column;
(iv) recovering a second flow through intermediate from the third chromatography column;
(v) passing the second flow through intermediate obtained from step (b)(iv) over a fourth chromatography column, wherein the fourth chromatography column is a ligand affinity column; and
(vi) recovering a purified protein eluate from the fourth chromatography column,
wherein the purified protein is a hemagglutinin (HA) protein derived from an influenza virus.

3. The method of claim 2, wherein the first chromatography column comprises a Butyl-S resin.

4. The method of claim 2, wherein the second chromatography column comprises a GP64 affinity resin.

5. The method of claim 2, wherein the third chromatography column comprises a trimethylaminoethyl (TMAE) resin.

6. The method of claim 2, wherein the fourth chromatography column comprises a lentil lectin affinity resin.

7. The method of claim 2, wherein the first chromatography column comprises a Butyl-S resin, the second chromatography column comprises a GP64 affinity resin, the third chromatography column comprises a trimethylaminoethyl (TMAE) resin, and the fourth chromatography column comprises a lentil lectin affinity resin.

8. The method of claim 2, wherein the purified protein is an HA protein derived from an influenza virus and the influenza virus is one or more influenza virus subtypes selected from the group consisting of: Influenza A Group 1, Influenza A Group 2, and Influenza B.

9. The method of claim 2, wherein the method for purifying the protein from the solution further comprises one or more filtration steps.

10. The method of claim 2, wherein step (a) further comprises a first filtration step, and wherein the first filtration step occurs after step (a)(ii).

11. The method of claim 2, wherein step (a) further comprises a first filtration step occurring after step (a)(ii), and wherein the first filtration step comprises tangential flow filtration.

12. The method of claim 10, wherein step (b) further comprises a second filtration step occurring after step (b)(iv) and a third filtration step occurring after step (b)(vi).

13. The method of claim 10, wherein step (b) further comprises a second filtration step occurring after step (b)(iv) and a third filtration step occurring after step (b)(vi), and wherein the second filtration step comprises viral filtration and the third filtration step comprises tangential flow filtration.

14. The method of claim 2, wherein step (a) further comprises a first filtration step occurring after step (a)(ii), and wherein step (b) further comprises a second filtration step occurring after step (b)(iv) and a third filtration step occurring after step (b)(vi).

15. The method of claim 2, wherein step (a) further comprises a first filtration step occurring after step (a)(ii), wherein step (b) further comprises a second filtration step occurring after step (b)(iv) and a third filtration step occurring after step (b)(vi), and wherein the second filtration step comprises viral filtration and the first and third filtration step comprises tangential flow filtration.

16. A purified respiratory syncytial virus (RSV) fusion (F) protein produced by a baculovirus expression system in cultured insect cells, wherein the purified influenza RSV F protein has a purity of greater than 85 wt %.

17. A method of purifying a protein from a solution comprising:
(a) a capture step comprising:
(i) passing the solution over a first chromatography column, wherein the first chromatography column is a hydrophobic interaction column; and
(ii) eluting a crude protein eluate from the first chromatography column; and
(b) a polish step comprising:
(i) passing the crude protein eluate obtained from step (a) over a second chromatography column, wherein the second chromatography column is a ligand affinity column;
(ii) recovering a first flow through intermediate from the second chromatography column;
(iii) passing the first flow through intermediate obtained from step (b)(ii) over a third chromatography column, wherein the third chromatography column is an anion exchange column;
(iv) recovering a second flow through intermediate from the third chromatography column;
(v) passing the second flow through intermediate obtained from step (b)(iv) over a fourth chromatography column, wherein the fourth chromatography column is a ligand affinity column; and
(vi) recovering a purified protein eluate from the fourth chromatography column,
wherein the purified protein is a fusion (F) protein derived from a respiratory syncytial virus (RSV).

18. The method of claim 17, wherein the first chromatography column comprises a Butyl-S resin.

19. The method of claim 17, wherein the second chromatography column comprises a GP64 affinity resin.

20. The method of claim 17, wherein the third chromatography column comprises a trimethylaminoethyl (TMAE) resin.

21. The method of claim 17, wherein the fourth chromatography column comprises a lentil lectin affinity resin.

22. The method of claim 17, wherein wherein the first chromatography column comprises a Butyl-S resin, the second chromatography column comprises a GP64 affinity resin, the third chromatography column comprises a trimethylaminoethyl (TMAE) resin, and the fourth chromatography column comprises a lentil lectin affinity resin.

23. The method of claim 17, wherein the purified protein is a fusion (F) protein derived from a respiratory syncytial virus (RSV) and the RSV is one or more RSV subtypes selected from the group consisting of: respiratory syncytial virus subtype A (RSV-A) and respiratory syncytial virus subtype B (RSV-B).

24. The method of claim 17, wherein the method for purifying the protein from the solution further comprises one or more filtration steps.

25. The method of claim 17, wherein step (a) further comprises a first filtration step, and wherein the first filtration step occurs after step (a)(ii).

26. The method of claim 17, wherein step (a) further comprises a first filtration step occurring after step (a)(ii), and wherein the first filtration step comprises tangential flow filtration.

27. The method of claim 25, wherein step (b) further comprises a second filtration step occurring after step (b)(iv) and a third filtration step occurring after step (b)(vi).

28. The method of claim 25, wherein step (b) further comprises a second filtration step occurring after step (b)(iv) and a third filtration step occurring after step (b)(vi), and wherein the second filtration step comprises viral filtration and the third filtration step comprises tangential flow filtration.

29. The method of claim 17, wherein step (a) further comprises a first filtration step occurring after step (a)(ii), and wherein step (b) further comprises a second filtration step occurring after step (b)(iv) and a third filtration step occurring after step (b)(vi).

30. The method of claim 17, wherein step (a) further comprises a first filtration step occurring after step (a)(ii), wherein step (b) further comprises a second filtration step occurring after step (b)(iv) and a third filtration step occurring after step (b)(vi), and wherein the second filtration step comprises viral filtration and the first and third filtration step comprises tangential flow filtration.
